(19) United States
(12) Patent Application Publication
(10) Pub. No.: US 2019/0070299 A1
(43) Pub. Date: Mar. 7, 2019

(54) DUAL-RATE RELEASE FORMULATION WITH HIGH DRUG LOADING

(71) Applicant: MATRIPHARM INTERNATIONAL INC., Laval (CA)

(72) Inventor: Tien Canh LE, Montreal (CA)

(21) Appl. No.: 16/079,009

(22) PCT Filed: Feb. 23, 2017
(86) PCT No.: PCT/CA2017/050224

§ 371 Date: Aug. 22, 2018

Related U.S. Application Data
(60) Provisional application No. 62/298,751, filed on Feb. 23, 2016.

(51) Int. Cl. A61K 47/38; A61K 47/18; A61K 31/167; A61K 31/496; A61K 9/20; A61P 31/04

(52) U.S. Cl. CPC A61K 47/38; A61K 47/183; A61K 31/167; A61K 47/02; A61K 9/2054; A61P 31/04; A61K 31/496

(57) ABSTRACT

The present document describes a pharmaceutical excipient composition comprising a functionalized anionic polysaccharide having carboxyl groups complexed with an amino acid-divalent cation complex, monolithic solid dosage forms for dual rate release of an active pharmaceutical ingredient, comprising the pharmaceutical excipient composition and active pharmaceutical ingredients, as well as processes for preparing the pharmaceutical excipient composition.

### CROSS-REFERENCE TO RELATED APPLICATIONS

This application claims priority of U.S. provisional patent application 62/298,751 filed on Feb. 23, 2016, the specification of which is hereby incorporated by reference.

### BACKGROUND

### (a) Field

The subject matter disclosed generally relates to a method to prepare a complex of anionic polysaccharide with an amino acid, preferably complexes of amino acid-calcium which is soluble in gastric fluid, but stable in intestinal fluid.

### (b) Related Prior Art

Anionic polysaccharides such as starch, cellulose, pectin, and others can be functionalized and used as excipient for dual rate release (DRR). However, starch is preferably used due to its low cost, biocompatibility and its non-toxicity. The functionalization of starch or other polysaccharides is preferably carboxylation such as carboxymethylation, carboxyethylation, succinylation, octenyl succinylation, acrylation, etc.

Generally, the process in the prior art is performed in an aqueous medium, methanol or ethanol, by etherification of the polysaccharide with sodium monochloroacetate, under alkaline conditions. In other processes, the functionalization was carried out in solvents such as methanol or ethanol in order to increase the degree of substitution (DS) of the polysaccharide. However, the obtained powder granules are fine and when compressed into tablets, they often break due to a lack of the cohesion. Furthermore, these excipients are used as disintegrating agents, as they swell when hydrated, and they cannot be used for DRR with high active pharmaceutical ingredient (API) loading.

Therefore, there is a need for additional excipients to mitigate the disadvantages of the excipients of the prior art.

### SUMMARY

According to an embodiment, there is provided a pharmaceutical excipient composition comprising a functionalized anionic polysaccharide having carboxyl groups complexed with an amino acid-divalent cation complex.

The amino acid may be chosen from lysine, arginine, histidine, or combinations thereof.

The divalent cation may be chosen from calcium, magnesium, zinc, aluminum, copper, or combinations thereof.

The functionalized anionic polysaccharide having carboxyl groups may be chosen from a starch, a cellulose, a chitosan, a guar gum, a gellan gum, a xanthan gum, an alginate, a pectate, an hyaluronate, a chondroitin, a carrageenan and combinations thereof.

The starch may be carboxymethyl starch.

The cellulose may be carboxymethyl cellulose.

The functionalized anionic polysaccharide having carboxyl groups has a molecular weight of about 40 to about 300 kDa, or from about 60 to about 160 kDa, or from about 80 to about 120 kDa, or about 100 kDa.

The functionalized anionic polysaccharide having carboxyl groups has a degree of substitution greater or equal to 0.15.

The functionalized anionic polysaccharide having carboxyl groups has a degree of substitution of 0.7.

According to another embodiment, there is provided a monolithic solid dosage form for dual rate release of an active pharmaceutical ingredient, comprising the pharmaceutical excipient composition of the present invention and the active pharmaceutical ingredient.

The monolithic solid dosage may further comprise a lubricating agent.

The lubricating agent may be chosen from magnesium stearate, calcium stearate, sodium stearate, sodium lauryl sulfate, mineral oil, polyethylene glycol, glyceryl palmitostearate a wax, glyceryl behenate, liquid paraffin.

The monolithic solid dosage form may further comprise a bulking agent.

The bulking agent may be microcrystalline cellulose.

The monolithic solid dosage form may further comprise a glidant.

The glidant may be a talc, a silicon dioxide, or combinations thereof.

The monolithic solid dosage form may further comprise a disintegrating agent.

The disintegrating agent may be cross-linked povidone, cross-linked sodium carboxymethyl cellulose, sodium starch glycolate, or combinations thereof.

The monolithic solid dosage form may further comprising a stabilizer.

The stabilizer may be carboxymethyl starch, an amino acid, or combinations thereof.

The amino acid may be arginine.

The active pharmaceutical ingredient may be acetaminophen.

The active pharmaceutical ingredient may be ciprofloxacin.

According to another embodiment, there is provided a method of treatment of a bacterial infection comprising administering to a subject in need thereof a therapeutic amount of a monolithic dosage form according to the present invention.

According to another embodiment, there is provided a use of a monolithic dosage form according to the present invention for the treatment of a bacterial infection in a subject in need thereof.

According to another embodiment, there is provided a monolithic dosage form according to the present invention for use in the treatment of a bacterial infection in a subject in need thereof.

According to another embodiment, there is provided a process for the preparation of an excipient, comprising:
a) mixing an amino acid with a divalent cation in an aqueous medium, at a ratio of about 10:1 to 1:1, to obtain an amino acid-divalent cation complex mixture;
b) introduce an alcohol in the amino acid-divalent cation complex mixture, to obtain a mixture of amino acid-divalent cation complex, water and alcohol;
c) introduce in the mixture of amino acid-divalent cation complex, water and alcohol a functionalized anionic polysaccharide having carboxyl groups, to initiate complexation for a time sufficient to obtain a functionalized anionic polysaccharide having carboxyl groups complexed with an amino acid-divalent cation complex.

The amino acid may be chosen from lysine, arginine, histidine, or combinations thereof.

The divalent cation may be chosen from calcium, magnesium, zinc, aluminum, copper, or combinations thereof.

The functionalized anionic polysaccharide having carboxyl groups may be chosen from a starch, a cellulose, a chitosan, a guar gum, a gellan gum, a xanthan gum, an alginate, a pectate, an hyaluronate, a chondroitin, a carrageenan and combinations thereof.

The starch may be carboxymethyl starch.

The cellulose may be carboxymethyl cellulose.

The functionalized anionic polysaccharide having carboxyl groups has a molecular weight of about 40 to about 300 kDa, or about 60 to about 160 kDa, or about 80 to about 120 kDa.

The functionalized anionic polysaccharide having carboxyl groups has a degree of substitution greater or equal to 0.15.

The following terms are defined below.

The term “amino acid” is intended to mean the organic compounds which contain amine (—NH2) and carboxylic acid (—COOH) functional groups, usually along with a side-chain specific to each amino acid. This include the 21 proteogenic alpha amino acids, alanine, valine, isoleucine, leucine, methionine, phenylalanine, tyrosine, tryptophan, serine, threonine, asparagine, glutamine, cysteine, selenocysteine, glycine, proline, arginine, histidine, lysine, aspartic acid, glutamic acid. This also includes beta, gamma, or delta amino acids suitable for the present invention.

The term «anionic polysaccharide» in intended to mean a polysaccharide having an overall negative charge. Examples of anionic polysaccharide suitable for use in the present invention include the starches, celluloses and other polysacharides that have been functionalized to bear negatively charged groups such as carboxyl groups such as chitosan, as well as guar gum, gellan gum, a xanthan gum, and the likes. In another embodiment, anionic polysaccharide of natural origin such as alginate (alginic acid), pectate (pectic acid), hyaluronate (hyaluronic acid or hyaluronan), chondroitin, gellan gum, carrageenan or combination thereof may be used.

As used herein, the term «functionalizing starch» or «functionalized starch», or «functionalizing cellulose» or «functionalized cellulose» is intended to mean functionalization that is not limited to the conversion of the native or modified starch or cellulose by carboxymethylation, but also includes possible functionalization (carboxylation) of other starch derivatives such as starch succinate (succinyl starch), hydroxypropyl starch, acetyl starch, hydroxypropyl methyl starch, acid modified starch, octenyl starch, pregelatinized starch or mixture thereof, or cellulose succinate (succinyl cellulose), hydroxypropyl cellulose, acetyl cellulose, hydroxypropyl methyl cellulose, acid modified cellulose, octenyl cellulose, pregelatinized cellulose or mixture thereof.

The term «functionalization» as used herein is intended to mean the addition by covalent bonds of carboxyl groups (or its derivatives) onto the starch chains. The functionalization can be (but is not limited to) the carboxylation (addition of carboxylate groups).

The term «carboxylation» as used herein is intended to mean the addition of carboxyl groups onto the polysaccharide macromolecule. Possible carboxylation includes but not limited to the carboxymethylation, carboxyethylation, succinylation, octenyl succinylation, acrylation, etc. According to a preferred embodiment, the carboxylation is a «carboxymethylation».

The term «degree of substitution» is intended to mean the average number of substituents per glucose unit (GU), the monomer unit of starch. Since each GU contains three hydroxyl groups, the DS can vary between 0-3. According to an embodiment of the present invention, the DS may be equal to or greater than 0.15.

The term “complexation” is intended to mean the process by which two or more ingredients are made to form a complex. According to the formulation of the present invention, a first complex is formed, in solution, between an amino acid and a divalent cation, which forms by nature of their opposed charges, and a second complex is formed between the functionalized anionic polysaccharide having carboxyl groups and the amino acid-divalent cation complex.

The term «composition» as used herein is intended to encompass a product comprising the specified ingredients in the specified amounts, as well as any product which results, directly or indirectly, from combination of the specified ingredients in the specified amounts. Such term in relation to pharmaceutical composition or other compositions in general, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions or other compositions in general of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

The term “hydrating agent” is intended to mean an agent that hydrates or favors the entry of water into the dosage form.

The term “buffering agent” is intended to mean an acid or base used to maintain the acidity (pH) of a solution near a chosen value after the addition of another acid or base. That is, the function of a buffering agent is to prevent a rapid change in pH when acids or bases are added to the solution.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates a schematic presentation of carboxymethylcellulose/arginine complex structure according to an embodiment of the present invention;

FIG. 2 illustrates a schematic presentation of the structure of arginine complexed with calcium ion according to an embodiment of the present invention;

FIG. 3 illustrates schematic presentation of (A-B) the probable structures of carboxymethylcellulose complexed with arginine-calcium complex according to an embodiment of the present invention;

FIG. 4 illustrates a FTIR spectra of carboxymethylcellulose (CMC) and complexes of CMC with Arginine (CMC/Arg) and with Arginine-Calcium (CMC/Arg-Ca) according to an embodiment of the present invention;

FIG. 5 illustrates the release kinetic profile of Acetaminophen (1000 mg) from a composition comprising the excipient according to an embodiment of the present invention;

FIG. 6 illustrates the release kinetic profiles of Acetaminophen (1000 mg) after 30 minutes in simulate gastric fluid, from a composition comprising the different quantities (30-90 mg) of excipient (CMC complexed with arginin-calcium complex) according to an embodiment of the present invention.

FIG. 7 illustrates the gastro retention technology using polymers possessing a highly-swollen capacity to prevent the tablet from passing through the pylorus and prolong the residence time in the stomach. During the stomach transit, the tablet releases continuously an appropriate quantity of active principle directly in the upper part of intestine

FIG. 8 illustrates the release kinestics of Ciprofloxacin (1000 mg) monolithic tablet formulated with xanthan gum complexed with calcium alone and with calcium-arginine in simulated gastric fluid according to an embodiment of the present invention.

### DETAILED DESCRIPTION

The present application discloses a new method for preparing a complex of anionic polysaccharide with an amino acid, preferably complexes of amino acid-calcium, which is soluble in gastric fluid, but stable in intestinal fluid. The complex can be used as excipient under monolithic tablet dosage form for delivering active pharmaceutical ingredients (API) in two different speeds (dual rate-release, DRR): i) an immediate and fast release of API in the stomach in order to provide effective concentrations required for a rapid relief; ii) a slow or sustained release of API in the intestine in order to maintain effective concentrations for a long period. The amino acid-calcium complex of the present invention displays reduced swelling and improved cohesion, which is suitable for DRR. Furthermore, this complex is able to support a high API loading which can reach up to 80% of the total composition.

In embodiments, there is disclosed a pharmaceutical excipient composition comprising a functionalized anionic polysaccharide having carboxyl groups complexed with an amino acid-divalent cation complex.

Anionic Polysaccharide Complexed with Amino Acid-Calcium Complexes

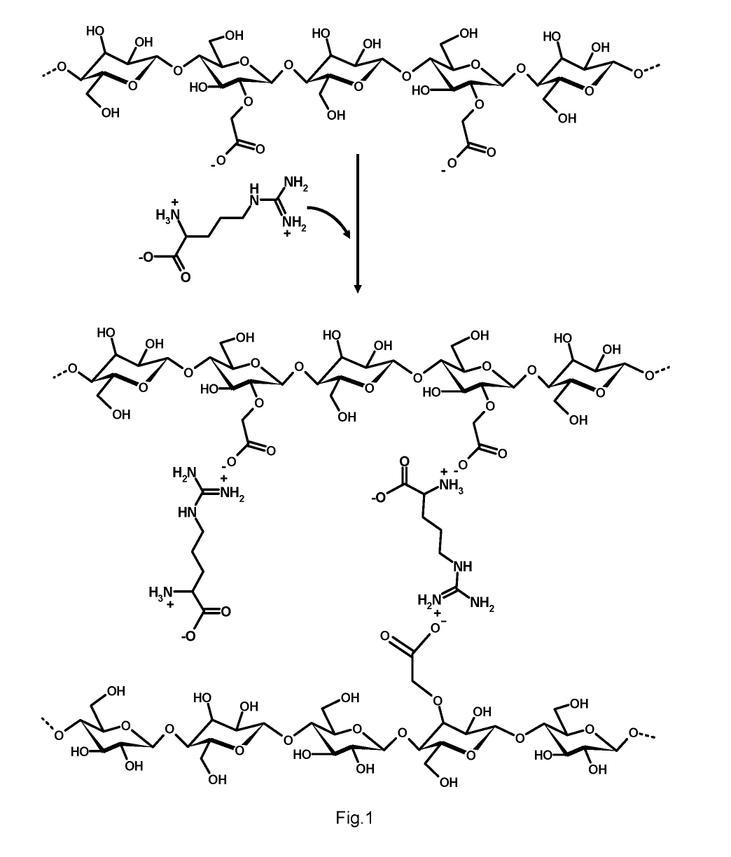

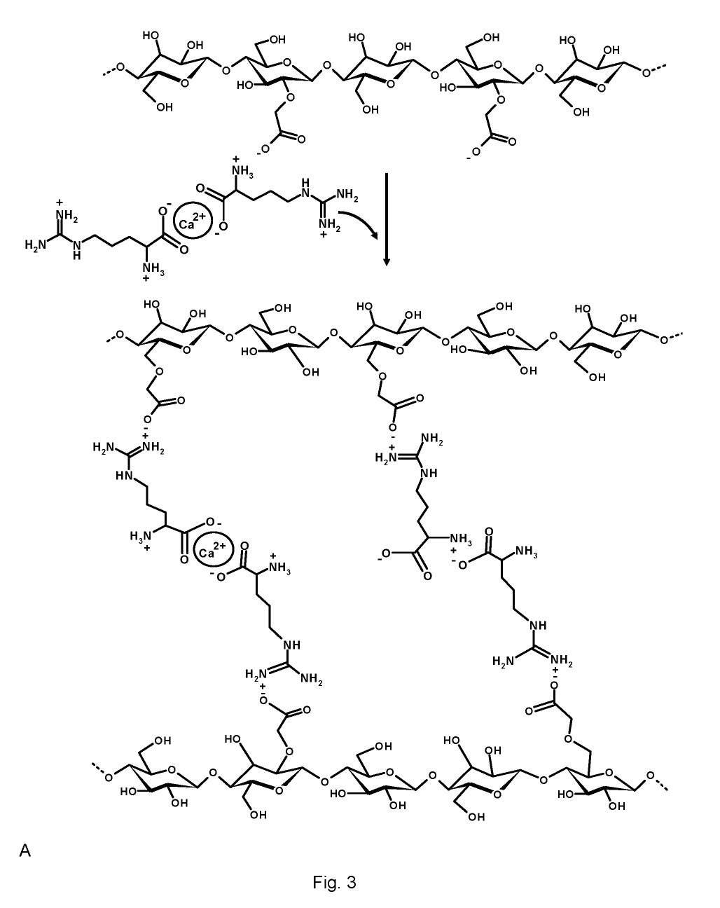

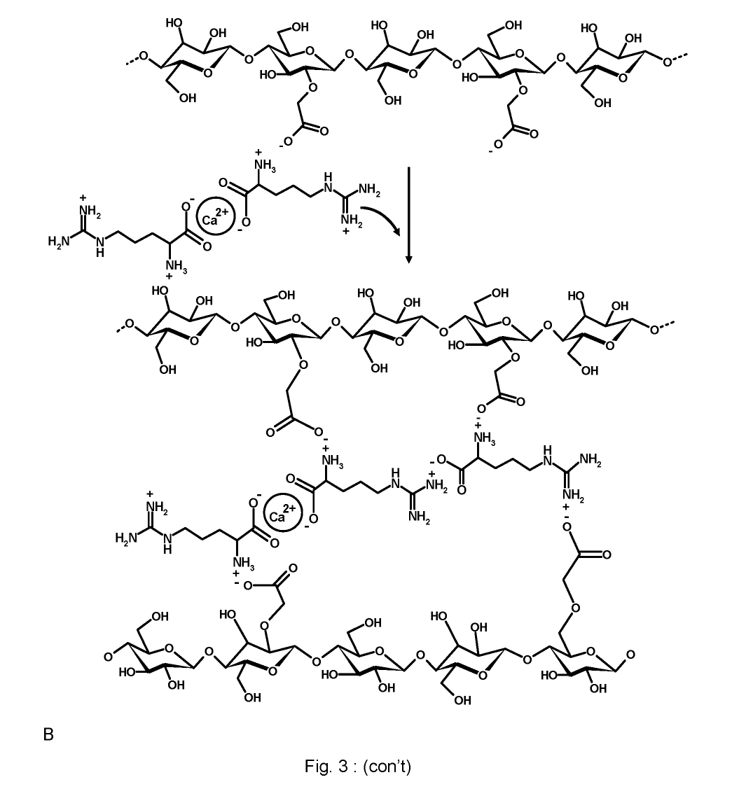

Anionic polysaccharides such as starches, celluloses, chitosans, that have been functionalized to bear negatively charged groups such as carboxyl groups, as well as guar gum, gellan gum, a xanthan gum, and the likes may be used in the present invention. Carboxymethylcellulose, carboxymethyl-starch, alginate, pectate (pectic acid), hyaluronate (hyaluronic acid or hyaluronan), chondroitin, gellan gum, carrageenan or combination thereof may also be used. These anionic polysaccharides possess negative charges and can complex with an amino acid. For example arginine (FIG. 1) or with a complex of arginine-calcium (FIGS. 3A and 3B). When formulated with carboxymethyl starch and free amino acid, this complex acts as mild disintegrating agent which is useful for dual-rate release (DRR): i) an immediate and fast release of active pharmaceutical ingredients (API) in the stomach in order to provide effective concentrations required for a rapid relief; ii) a slow or sustained release of API in the intestine in order to maintain effective concentrations for a long period.

According to an embodiment of the present invention, the anionic polysaccharides such as carboxymethyl cellulose (CMC) and carboxymethyl starch (CMS) are preferably used due to their low cost, biocompatibility and availability in the market. According to the molecular weight (MW) and the degree of substitution (DS) of these anionic polysaccharides, the solubility of complex of anionic polysaccharide formed with arginine or arginine-calcium in the gastric fluid is different and can significantly modulate the speed of the immediate and fast release. In embodiments, the lower the MW of the anionic polysaccharides, the faster the release of the API will be, and the higher the MW, the slower the release of the API will be. Similarly, in embodiments, the lower the DS of the anionic polysaccharides, the faster the release of the API will be, and the higher the DS, the slower the release of the API will be. According to some embodiments, the MW of the anionic polysaccharide may be from about 40 to about 300, or from about 60 to about 160, or from about 80 to about 120 kDa, or from about 85 to about 120 kDa, or from about 90 to about 120 kDa, or from about 95 to about 120 kDa, or from about 100 to about 120 kDa, or from about 105 to about 120 kDa, or from about 110 to about 120 kDa, or from about 115 to about 120 kDa, and the DS≥0.15, and from about 0.4 to about 1.5, or from about 0.4 to about 1.4, or from about 0.4 to about 1.3, or from about 0.4 to about 1.2, or from about 0.4 to about 1.1, or from about 0.4 to about 1.0, or from about 0.4 to about 0.9, or from about 0.4 to about 0.8, or from about 0.4 to about 0.7, or from about 0.4 to about 0.6, or from about 0.4 to about 0.5, or from about 0.5 to about 1.5, or from about 0.5 to about 1.4, or from about 0.5 to about 1.3, or from about 0.5 to about 1.2, or from about 0.5 to about 1.1, or from about 0.5 to about 1.0, or from about 0.5 to about 0.9, or from about 0.5 to about 0.8, or from about 0.5 to about 0.7, or from about 0.5 to about 0.6, or from about 0.6 to about 1.5, or from about 0.6 to about 1.4, or from about 0.6 to about 1.3, or from about 0.6 to about 1.2, or from about 0.6 to about 1.1, or from about 0.6 to about 1.0, or from about 0.6 to about 0.9, or from about 0.6 to about 0.8, or from about 0.6 to about 0.7, or from about 0.7 to about 1.5, or from about 0.7 to about 1.4, or from about 0.7 to about 1.3, or from about 0.7 to about 1.2, or from about 0.7 to about 1.1, or from about 0.7 to about 1.0, or from about 0.7 to about 0.9, or from about 0.7 to about 0.8, or from about 0.8 to about 1.5, or from about 0.8 to about 1.4, or from about 0.8 to about 1.3, or from about 0.8 to about 1.2, or from about 0.8 to about 1.1, or from about 0.8 to about 1.0, or from about 0.8 to about 0.9, or from about 0.9 to about 1.5, or from about 0.9 to about 1.4, or from about 0.9 to about 1.3, or from about 0.9 to about 1.2, or from about 0.9 to about 1.1, or from about 0.9 to about 1.0, or from about 1.0 to about 1.5, or from about 1.0 to about 1.4, or from about 1.0 to about 1.3, or from about 1.0 to about 1.2, or from about 1.0 to about 1.1, or from about 1.1 to about 1.5, or from about 1.1 to about 1.4, or from about 1.1 to about 1.3, or from about 1.1 to about 1.2, or from about 1.2 to about 1.5, or from about 1.2 to about 1.4, or from about 1.2 to about 1.3, or from about 1.3 to about 1.5, or from about 1.3 to about 1.4, or from about 1.4 to about 1.5, or about 0.4, 0.5, 0.6, 0.7, 0.8, 0.9, 1.0, 1.1, 1.2, 1.3, 1.4, and 1.5.

In embodiments, any amino acids could be used for the complexation with the anionic polysaccharide. However, the anionic polysaccharide/amino acid complex is stable in gastric acid fluid, favoring the immediate release of active principle by erosion and without altered the integrity of the tablet.

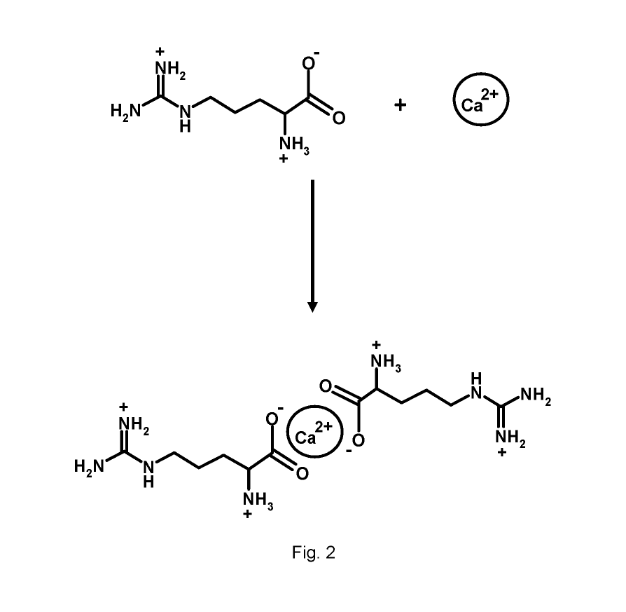

Therefore, according to an embodiment, to improve the initial fast release, the amino acid is first complexed with calcium to form a complex of amino acid-calcium (FIG. 2) prior to complexation of the amino acid-calcium with the anionic polysaccharide. This amino acid-calcium complex possesses a high hydration capacity in gastric acid fluid (pH 1.0-4.0) and promotes thus the immediate release of API in the stomach. However, it becomes stable in the intestinal fluid (pH>6.0) and limits the API release rate to a slower and/or sustained release rate. In embodiments of the present invention, there is no significant complexation of calcium ion with the anionic polysaccharide, otherwise an insoluble aggregation would be generated, which could not be used as excipient for DRR.

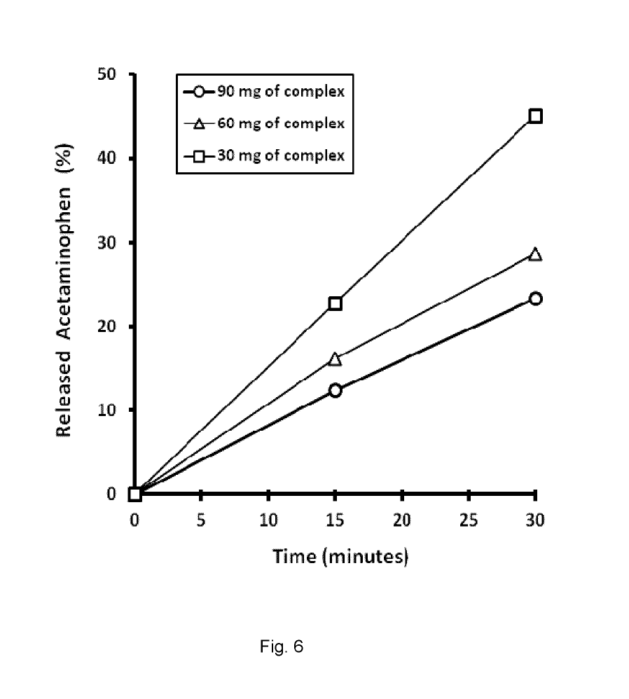

According to an embodiment, when formulated with CMS and free amino acid, the complex of anionic polysaccharide/amino acid-calcium presents surprising behavior (FIG. 6): in low quantities, the complex of anionic polysaccharide/amino acid-calcium behaves as a disintegrating agent, whereas at high quantities, it acts as a slow release agent. Therefore, according to another embodiment, another aspect of the present invention which is relevant in the present application is the possibility to adjust the amount of API released by using an appropriate quantity of the complex of anionic polysaccharide/amino acid-calcium. Therefore, according to embodiments, the ratio of the complex of anionic polysaccharide/amino acid-calcium to API (i.e. complex:API ratio) may be from about 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, or from about 1:25 to about 1:4, or from about 1:24 to about 1:4, or from about 1:23 to about 1:4, or from about 1:22 to about 1:4, or from about 1:21 to about 1:4, or from about 1:20 to about 1:4, or from about 1:19 to about 1:4, or from about 1:18 to about 1:4, or from about 1:17 to about 1:4, or from about 1:16 to about 1:4, or from about 1:15 to about 1:4, or from about 1:14 to about 1:4, or from about 1:13 to about 1:4, or from about 1:12 to about 1:4, or from about 1:11 to about 1:4, or from about 1:10 to about 1:4, or from about 1:9 to about 1:4, or from about 1:8 to about 1:4, or from about 1:7 to about 1:4, or from about 1:6 to about 1:4, or from about 1:5 to about 1:4, or from about 1:25 to about 1:5, or from about 1:24 to about 1:5, or from about 1:23 to about 1:5, or from about 1:22 to about 1:5, or from about 1:21 to about 1:5, or from about 1:20 to about 1:5, or from about 1:19 to about 1:5, or from about 1:18 to about 1:5, or from about 1:17 to about 1:5, or from about 1:16 to about 1:5, or from about 1:15 to about 1:5, or from about 1:14 to about 1:5, or from about 1:13 to about 1:5, or from about 1:12 to about 1:5, or from about 1:11 to about 1:5, or from about 1:10 to about 1:5, or from about 1:9 to about 1:5, or from about 1:8 to about 1:5, or from about 1:7 to about 1:5, or from about 1:6 to about 1:5, or from about 1:25 to about 1:6, or from about 1:24 to about 1:6, or from about 1:23 to about 1:6, or from about 1:22 to about 1:6, or from about 1:21 to about 1:6, or from about 1:20 to about 1:6, or from about 1:19 to about 1:6, or from about 1:18 to about 1:6, or from about 1:17 to about 1:6, or from about 1:16 to about 1:6, or from about 1:15 to about 1:6, or from about 1:14 to about 1:6, or from about 1:13 to about 1:6, or from about 1:12 to about 1:6, or from about 1:11 to about 1:6, or from about 1:10 to about 1:6, or from about 1:9 to about 1:6, or from about 1:8 to about 1:6, or from about 1:7 to about 1:6, or from about 1:25 to about 1:7, or from about 1:24 to about 1:7, or from about 1:23 to about 1:7, or from about 1:22 to about 1:7, or from about 1:21 to about 1:7, or from about 1:20 to about 1:7, or from about 1:19 to about 1:7, or from about 1:18 to about 1:7, or from about 1:17 to about 1:7, or from about 1:16 to about 1:7, or from about 1:15 to about 1:7, or from about 1:14 to about 1:7, or from about 1:13 to about 1:7, or from about 1:12 to about 1:7, or from about 1:11 to about 1:7, or from about 1:10 to about 1:7, or from about 1:9 to about 1:7, or from about 1:8 to about 1:7, or from about 1:25 to about 1:8, or from about 1:24 to about 1:8, or from about 1:23 to about 1:8, or from about 1:22 to about 1:8, or from about 1:21 to about 1:8, or from about 1:20 to about 1:8, or from about 1:19 to about 1:8, or from about 1:18 to about 1:8, or from about 1:17 to about 1:8, or from about 1:16 to about 1:8, or from about 1:15 to about 1:8, or from about 1:14 to about 1:8, or from about 1:13 to about 1:8, or from about 1:12 to about 1:8, or from about 1:11 to about 1:8, or from about 1:10 to about 1:8, or from about 1:9 to about 1:8, or from about 1:25 to about 1:9, or from about 1:24 to about 1:9, or from about 1:23 to about 1:9, or from about 1:22 to about 1:9, or from about 1:21 to about 1:9, or from about 1:20 to about 1:9, or from about 1:19 to about 1:9, or from about 1:18 to about 1:9, or from about 1:17 to about 1:9, or from about 1:16 to about 1:9, or from about 1:15 to about 1:9, or from about 1:14 to about 1:9, or from about 1:13 to about 1:9, or from about 1:12 to about 1:9, or from about 1:11 to about 1:9, or from about 1:10 to about 1:9, or from about 1:25 to about 1:10, or from about 1:24 to about 1:10, or from about 1:23 to about 1:10, or from about 1:22 to about 1:10, or from about 1:21 to about 1:10, or from about 1:20 to about 1:10, or from about 1:19 to about 1:10, or from about 1:18 to about 1:10, or from about 1:17 to about 1:10, or from about 1:16 to about 1:10, or from about 1:15 to about 1:10, or from about 1:14 to about 1:10, or from about 1:13 to about 1:10, or from about 1:12 to about 1:10, or from about 1:11 to about 1:10, or from about 1:25 to about 1:11, or from about 1:24 to about 1:11, or from about 1:23 to about 1:11, or from about 1:22 to about 1:11, or from about 1:21 to about 1:11, or from about 1:20 to about 1:11, or from about 1:19 to about 1:11, or from about 1:18 to about 1:11, or from about 1:17 to about 1:11, or from about 1:16 to about 1:11, or from about 1:15 to about 1:11, or from about 1:14 to about 1:11, or from about 1:13 to about 1:11, or from about 1:12 to about 1:11, or from about 1:25 to about 1:12, or from about 1:24 to about 1:12, or from about 1:23 to about 1:12, or from about 1:22 to about 1:12, or from about 1:21 to about 1:12, or from about 1:20 to about 1:12, or from about 1:19 to about 1:12, or from about 1:18 to about 1:12, or from about 1:17 to about 1:12, or from about 1:16 to about 1:12, or from about 1:15 to about 1:12, or from about 1:14 to about 1:12, or from about 1:13 to about 1:12, or from about 1:25 to about 1:13, or from about 1:24 to about 1:13, or from about 1:23 to about 1:13, or from about 1:22 to about 1:13, or from about 1:21 to about 1:13, or from about 1:20 to about 1:13, or from about 1:19 to about 1:13, or from about 1:18 to about 1:13, or from about 1:17 to about 1:13, or from about 1:16 to about 1:13, or from about 1:15 to about 1:13, or from about 1:14 to about 1:13, or from about 1:25 to about 1:14, or from about 1:24 to about 1:14, or from about 1:23 to about 1:14, or from about 1:22 to about 1:14, or from about 1:21 to about 1:14, or from about 1:20 to about 1:14, or from about 1:19 to about 1:14, or from about 1:18 to about 1:14, or from about 1:17 to about 1:14, or from about 1:16 to about 1:14, or from about 1:15 to about 1:14, or from about 1:25 to about 1:15, or from about 1:24 to about 1:15, or from about 1:23 to about 1:15, or from about 1:22 to about 1:15, or from about 1:21 to about 1:15, or from about 1:20 to about 1:15, or from about 1:19 to about 1:15, or from about 1:18 to about 1:15, or from about 1:17 to about 1:15, or from about 1:16 to about 1:15, or from about 1:25 to about 1:16, or from about 1:24 to about 1:16, or from about 1:23 to about 1:16, or from about 1:22 to about 1:16, or from about 1:21 to about 1:16, or from about 1:20 to about 1:16, or from about 1:19 to about 1:16, or from about 1:18 to about 1:16, or from about 1:17 to about 1:16, or from about 1:25 to about 1:17, or from about 1:24 to about 1:17, or from about 1:23 to about 1:17, or from about 1:22 to about 1:17, or from about 1:21 to about 1:17, or from about 1:20 to about 1:17, or from about 1:19 to about 1:17, or from about 1:18 to about 1:17, or from about 1:25 to about 1:18, or from about 1:24 to about 1:18, or from about 1:23 to about 1:18, or from about 1:22 to about 1:18, or from about 1:21 to about 1:18, or from about 1:20 to about 1:18, or from about 1:19 to about 1:18, or from about 1:25 to about 1:19, or from about 1:24 to about 1:19, or from about 1:23 to about 1:19, or from about 1:22 to about 1:19, or from about 1:21 to about 1:19, or from about 1:20 to about 1:19, or from about 1:25 to about 1:20, or from about 1:24 to about 1:20, or from about 1:23 to about 1:20, or from about 1:22 to about 1:20, or from about 1:21 to about 1:20, or from about 1:25 to about 1:21, or from about 1:24 to about 1:21, or from about 1:23 to about 1:21, or from about 1:22 to about 1:21, or from about 1:25 to about 1:22, or from about 1:24 to about 1:22, or from about 1:23 to about 1:22, or from about 1:25 to about 1:23, or from about 1:24 to about 1:23 or from about 1:25 to about 1:24.

Although all the amino acids can be used for complexing with anionic polysaccharides, charged amino acids are preferred, particularly negatively charged amino acids such as arginine, lysine, and histidine.

### Preparation of the Anionic Polysaccharide/Amino Acid-Calcium Complex

According to another embodiment, anionic polysaccharide such as CMC and amino acid-calcium such as arginine-calcium are preferably used. The complexation of CMC with arginine-calcium complex can be performed in two steps.

The first step involves preparing the complex of arginine with calcium ion. The preparation is mainly carried out in an aqueous medium by mixing arginine with calcium chloride. The ratio of arginine-calcium can vary from 10:1 to 1:1 (w/w).

The second step is to complex the CMC with the complex of arginine-calcium obtained above. The medium of complexation is essentially in hydroalcoholic (i.e. water/ethanol) solution. Initially, the ratio of water/alcohol is about 50:50 (v/v) and, as water is added during the reaction, the ratio of water/alcohol at the end the reaction can reach up to 90:10 (v/v).

The complex obtained as final product becomes insoluble and is easy to separate from the medium by decantation or filtration. Furthermore, the complexation reaction is rapid and low cost and no heating is necessary.

### Monolithic Tablet Formulation for Dual Rate Release

According to an embodiment, there is disclosed a dual release rate formulation comprising:
a) Carboxymethylcellulose/arginine-calcium complex of the present invention (excipient for DRR);
b) Hydrophilic polymer, such as Carboxymethyl starch or polivinyl pyrrolidone derivatives (hydrating agent);
c) An amino acid, such as free arginine (buffering agent);
d) Other ingredients could be added in the formulation such as lubricating agents such as stearic acid or its salts, (e.g. magnesium stearate or calcium stearate), sodium lauryl sulfate, mineral oil, polyethylene glycol, glyceryl palmitostearate, glyceryl behenate;
e) Glidants, such as talc and silicone dioxide;
f) Disintegrating agents, for example cross-linked sodium carboxymethyl cellulose (croscarmellose), cross-linked polyvinylpyrrolidone (Crospovidone™), sodium starch glycolate (Explotab®); and
g) An API.

The present invention will be more readily understood by referring to the following examples which are given to illustrate the invention rather than to limit its scope.

### Example 1

### Preparation of Carboxymethylcellulose/Arginine-Calcium Complex

### 1-1 Preparation of Arginine-Calcium Complex

An amount of 15 g of arginine (free base) is solubilized in 200 mL of deionized water under mild stirring. When the arginine is completely dissolved in the solution, an amount of 5 g of calcium chloride dihydrate is introduced in the arginine solution until obtained a clear solution. These conditions represent a molar ratio of arginine:calcium of 2.5:1.0, which means that arginine is in excess. The pH of arginine free base is about 10, and therefore the majority of carboxyl group are negatively charged, which implies that the reaction is near 100% complete and that no significant amount of calcium remains uncomplexed with arginine. Finally, a volume of 200 mL of ethanol is added in the arginine-calcium complex, always under mild stirring for 10 minutes.

1-2 Complexation of CMC with Arginine-Calcium Complex

An amount of 20 g of CMC (MW=100 kDa, and DS=0.7) is slowly introduced in the solution of arginine-calcium complex (obtained above) under vigorous stirring. After 10 minutes of dispersion, a volume of 600 mL of deionized water is slowly added and the reaction of complexation is continued for at least 2 h.

Finally, the complex powder is collected by decantation (or by filtration) and washed with 500 mL of absolute ethanol before incubation in an oven for at least 10 h at 40° C.

### 1-3 FTIR Analysis

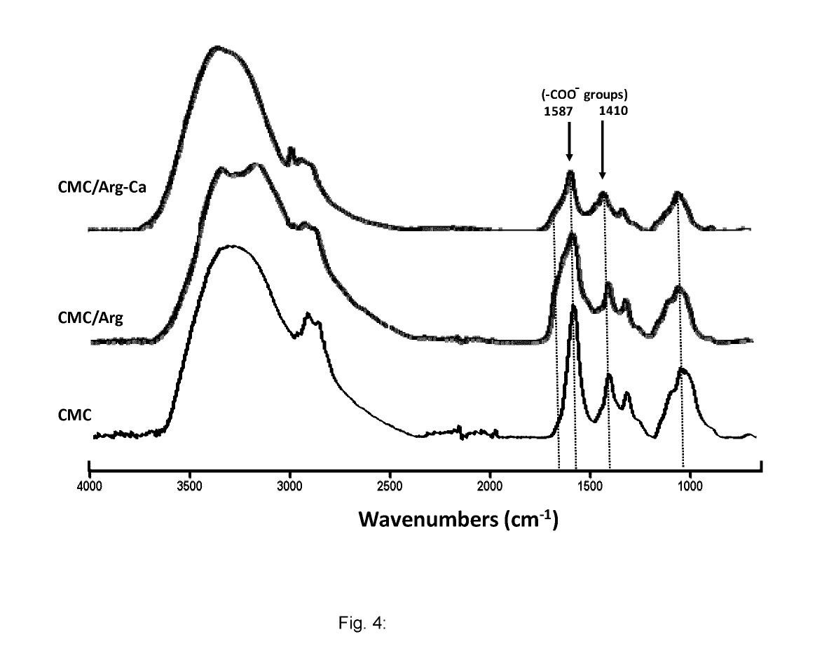

FTIR analysis (FIG. 4) shows for CMC absorption bands at 1587 and 1410 cm⁻¹ assigned to carboxylate anions (asymmetric and symmetric stretching vibrations). When CMC complexed with arginine, a moderate decrease of the intensities for absorption bands at 1587 and 1410 cm⁻¹ was observed and probably due to interactions between carboxylate groups, not only from CMC, but also carboxylate group from arginine, with amine groups from arginine.

Similar observation for the CMC/arginine-calcium complex is noticed, but the intensities of the absorption bands assigned for carboxylate groups are markedly reduced. This reduction in intensity is probably due to interaction mainly of carboxylate group from CMC with amine group from arginine.

### Example 2

### Preparation of Carboxymethyl-Starch/Arginine-Calcium Complex

The preparation of the carboxymethyl-starch/arginine-calcium complex is conducted under similar conditions, as described previously above in Example 1, except the carboxymethyl-starch (MW=150 kDa and DS=0.4) has been used instead of carboxymethylcellulose.

### Example 3

### Preparation of Alginate/Arginine-Calcium Complex

The preparation of the alginate/arginine-calcium complex is conducted under similar conditions, as described previously above in Example 1, except the alginate has been used instead of carboxymethylcellulose.

### Example 4

### Preparation of Carboxymethylcellulose/Lysine-Calcium Complex

The preparation of the carboxymethylcellulose/(lysine-calcium) complex is conducted under similar conditions, as described previously above in Example 1 (section 1-1), except the lysine has been used instead of arginine.

### Example 5

### Preparation of Carboxymethylcellulose/(Arginine/Lysine)-Calcium Complex

The preparation of the carboxymethylcellulose/(arginine/lysine)-calcium complex is conducted under similar conditions, as described previously above in Example 1 (section 1-1), except an equimolar mixture of arginine and lysine has been used instead of arginine.

### Example 6

### Acetaminophen Dual-Rate Release Kinetic Study Used Carboxymethyl Cellulose/Arginine-Calcium Complex as Excipient

TABLE 1
6-1 Formulation for Monolithic Tablet Dosage Form

| | Quantity | |
|---|---|---|
| Ingredient | (mg) | % |
| Acetaminophen | 1000 | 76.9 |
| Carboxymethyl starch | 200 | 15.4 |
| Carboxymethylcellulose/Arginine-Calcium Complex | 40 | 3.1 |
| Arginine | 40 | 3.1 |
| Magnesium stearate | 20 | 1.5 |
| Total | 1300 | 100.0 |

MW of Carboxymethylcellulose = 100 kDa, and DS = 0.7)

### 6-2 Preparation of Monolithic Tablet

Monolithic tablets containing 1000 mg (˜77%) of acetaminophen as a tracer were obtained by direct compression of powders (2.3 T/cm² in a Carver hydraulic press).

### 6-3 Dissolution Assay

The dissolution kinetic assay is followed with a Distek apparatus according to the paddle method from USP-32, with slight modification. Indeed, the monolithic tablets are placed in 750 mL of simulated gastric fluid (SGF, pH 1.5) during 30 minutes, at 37° C. Thereafter, a volume of 250 mL of tribasic sodium phosphate 0.20 M is added directly in SGF to neutralize the gastric acidity for pH values about of 6.8-7.0 which are constituted the simulated intestinal fluid (SIF).

At different intervals (15, 30 minutes for SGF and 30 minutes for SIF), an aliquot of 1 mL of dissolution fluid is withdrawn from the dissolution media, filtered and properly diluted (approximately 1/50). The absorbency is measured with an UV (Lambda-40 Spectrometer, Perkin Elmer) at 247 nm.

### 6-4 Results

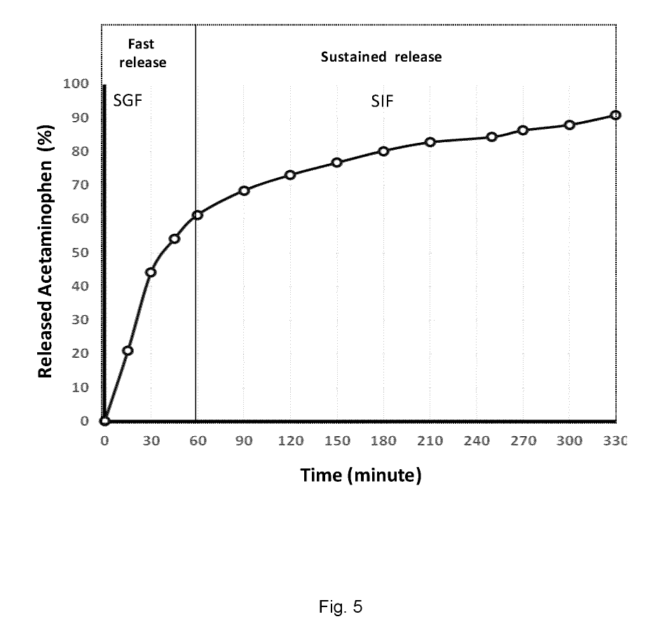

Data analysis shows that the immediate release of Acetaminophen is about of 60% (600 mg of Acetaminophen) after 30 minutes in SGF and 15 minutes in SIF. The remained quantity of Acetaminophen is slowly released in SIF for a period approximately 5 h or more (FIG. 5).

### Example 7

### Formulation of Ciprofloxacin Extended Release

Ciprofloxacin is an insoluble antibiotic, a synthetic broad-spectrum antimicrobial agent for oral administration. Ciprofloxacin is difficult to formulate due to its solubility. As a hydrochloride salt form, it is soluble in acidic medium such as in the gastric fluid (pH 0.1 N) or in the intestine upper part (small intestine including duodenum, jejunum and ileum where their pH is about 4.5-5.5). Ciprofloxacin becomes insoluble in alkaline medium, particularly in intestinal fluid where it aggregates and forms particles, and results in reduced bioavailability.

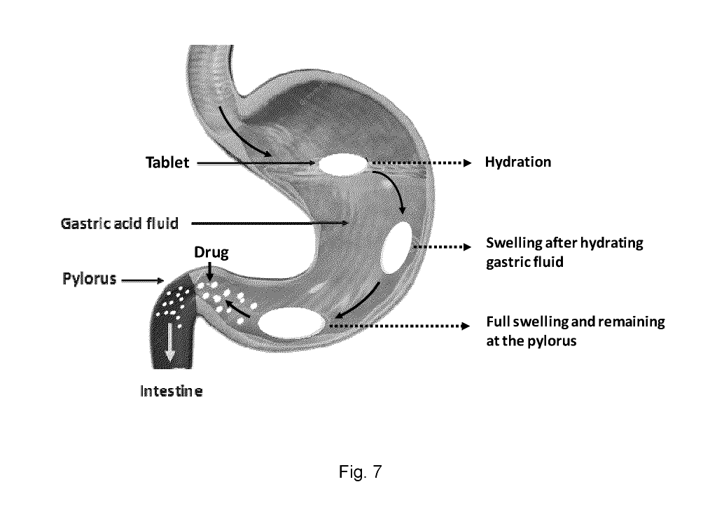

For this reason, Ciprofloxacin is generally formulated under the Gastro-Retention Dosage Form (GRDF). This technology consists in using polymers possessing a highly-swollen capacity to prevent the tablet from passing through the pylorus and prolong the residence time in the stomach. During the stomach transit, the tablet releases continuously an appropriate quantity of Ciprofloxacin directly in the upper part of intestine (FIG. 7).

This GRDF technology is characterized by 1) a longer retention time of the tablets in the stomach, where Ciprofloxacin is soluble, and ensures a bioavailability and 2) the release of Ciprofloxacin occurs locally and continuously from the stomach to the upper part of the intestine that is the main absorption site for Ciprofloxacin.

Several Bayer™ formulations currently in the market are based on this technology. By example, CIPRO® XR (ciprofloxacin*extended-release tablets). CIPRO XR® Tablets are coated, bilayer tablets consisting of an immediate-release layer and an erosion-matrix type controlled-release layer. The tablets contain a combination of two types of ciprofloxacin drug substance, ciprofloxacin hydrochloride and ciprofloxacin betaine (base).

Ciprofloxacin hydrochloride is 1-cyclopropyl-6-fluoro-1, 4-dihydro-4-oxo-7-(1-piperazinyl)-3-quinoline carboxylic acid hydrochloride. It is provided as a mixture of the monohydrate and the sesquihydrate. The empirical formula of the monohydrate is C₁₇H₁₈FN₃O₃.HCl.H₂O and its molecular weight is 385.8. The empirical formula of the sesquihydrate is C₁₇H₁₈FN₃O₃.HCl.1.5 H₂O and its molecular weight is 394.8. The drug substance is a faintly yellowish to light yellow crystalline substance. The chemical structure of the monohydrate is as follows:

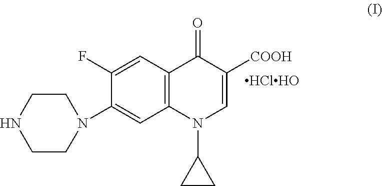 [SMILES: O=C(O)c1cn(C2CC2)c2cc(N3CCNCC3)c(F)cc2c1=O]

Ciprofloxacin betaine is 1-cyclopropyl-6-fluoro-1, 4-dihydro-4-oxo-7-(1-piperazinyl)-3-quinoline carboxylic acid. As a hydrate, its empirical formula is C₁₇H₁₈FN₃O₃.3.5 H2O and its molecular weight is 394.3. It is a pale yellowish to light yellow crystalline substance and its chemical structure is as follows:

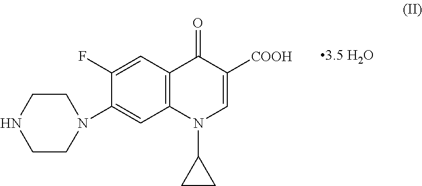 [SMILES: O=C(O)c1cn(C2CC2)c2cc(N3CCNCC3)c(F)cc2c1=O]

CIPRO XR® tablets are available as 500 mg (ciprofloxacin equivalent) tablets strengths. CIPRO XR® tablets are nearly white to slightly yellowish, film-coated, oblong-shaped tablets. Each CIPRO XR® 500 mg tablet contains 500 mg of ciprofloxacin as ciprofloxacin HCl (287.5 mg, calculated as ciprofloxacin on the dried basis) and ciprofloxacin (212.6 mg, calculated on the dried basis).

In the present embodiment, the use of carboxylate polymer complexed with Calcium Arginine is used as excipient to formulate Ciprofloxacin HCl under monolithic tablet dosage form for controlled release. The kinetic release profile of this tablet dosage form may change as a function of pH of the (carboxylate polymer/Ca-Arginine) complex. For example, at low pH values (i.e. <5.5), a DRR of Ciprofloxacin is noticed whereas with neutral or higher pH values, a sustained release is observed. This parameter is important to adjust the fast release of active principle.

The advantages of the present invention are that it is easy and simple to manufacture, since monolithic tablet are simply prepared by mixing the active principle(s) with excipient powders for direct compression, the low cost compared to bilayer tablet, and only a salt form of the active principle is used, such as ciprofloxacin hydrochloride, instead of a mixture of ciprofloxacin hydrochloride and ciprofloxacin betaine.

In the present example, xanthan gum is used because xanthan is a carboxylate polymer which is composed of pentasaccharide repeat units including glucose, manose and glucuronate in the molar ratio 2:2:1. Xanthan gum is stable and swells significantly in aqueous medium. Alternatives are CMC, CMS, and alginates.

TABLE 2
formulation according to the present invention

| | Quantity | |
|---|---|---|
| Ingredient | (mg) | Percentage |
| Ciprofloxacin HCl | 1000.0 | 75.8 |
| Xanthan/Ca-Arg complex (pH | 90.0 | 6.8 |
| 4.5) | | |
| Crospovidone | 90.0 | 6.8 |
| Polyethylen glycol 3350 | 80.0 | 6.1 |
| Microcrystalline cellulose | 40.0 | 3.0 |
| Magnesium stearate | 20.0 | 1.5 |
| | 1320.0 | 100.0 |

TABLE 3
Comparative composition

| | Quantity (mg) | Percentage (%) |
|---|---|---|
| Ciprofloxacin HCl | 1000.0 | 75.8 |
| Xanthan/Ca | 90.0 | 6.8 |
| Crospovidone | 90.0 | 6.8 |
| Polyethylen glycol 3350 | 80.0 | 6.1 |
| Microcrystalline cellulose | 40.0 | 3.0 |
| Magnesium stearate | 20.0 | 1.5 |
| | 1320.0 | 100.0 |

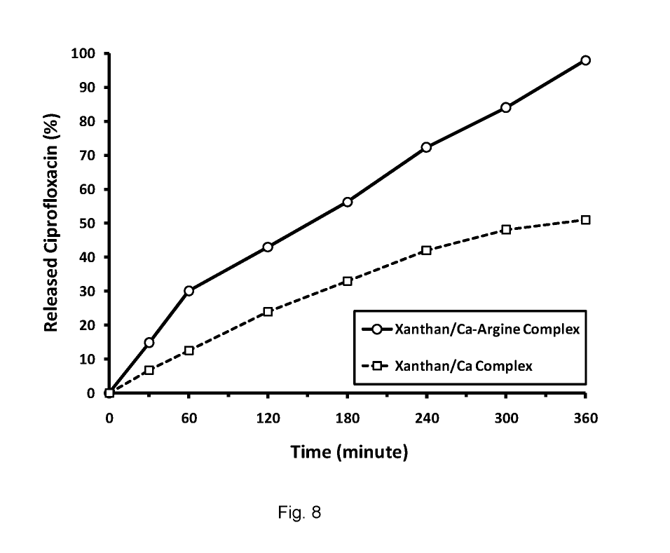

FIG. 8 illustrates the dissolution release profile of ciprofloxacin (1000 mg) monolithic tablet formulated with xanthan gum complexed with calcium alone and with Ca-Arginine according to the present invention, in simulated gastric fluid.

Release profiles of ciprofloxacin HCl from the matrices were performed in a dissolution apparatus 2 at 50 rpm, 37.0±0.5° C., using 900 mL of USP simulated gastric fluid without pepsine as dissolution medium (pH=1.2). Samples of 1.0 ml were taken at predetermined time intervals and after properly diluted with medium (SGF) solution, the amount of released ciprofloxacin was measured spectrophotometrically at maximum absorbance wavelength, 276 nm.

The result shows that a DRR is observed for Ciprofloxacin formulated with xanthan gum complexed with Ca-Arginine under monolithic tablet dosage form, whereas no DRR is noticed for Ciprofloxacin formulated with xanthan complex with Calcium alone.

In fact, the xanthan/calcium complex tablet favors the formation of a stable gel-like structure in gastric acidity, thus sustaining the release of ciprofloxacin. On the opposite, no formation of gel-like character for xanthan complexed with calcium-arginine is observed, but the tablet is swollen and partially disintegrated to provide an initial fast release. When the tablet is completely hydrated, a ciprofloxacin release by erosion is observed.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A pharmaceutical excipient composition comprising a functionalized anionic polysaccharide having carboxyl groups complexed with an amino acid-divalent cation complex.

2. The pharmaceutical excipient composition of claim 1, wherein said amino acid is chosen from lysine, arginine, histidine, or combinations thereof.

3. The pharmaceutical excipient composition of claim 1, wherein a divalent cation is chosen from calcium, magnesium, zinc, aluminum, copper, or combinations thereof.

4. The pharmaceutical excipient composition of claim 1, wherein said functionalized anionic polysaccharide having carboxyl groups is chosen from a starch, a cellulose, a chitosan, a guar gum, a gellan gum, a xanthan gum, an alginate, a pectate, an hyaluronate, a chondroitin, a carrageenan and combinations thereof.

5. The pharmaceutical excipient composition of claim 4, wherein said starch is carboxymethyl starch, and said cellulose is carboxymethyl cellulose.

6. (canceled)

7. The pharmaceutical excipient composition of claim 1, wherein said functionalized anionic polysaccharide having carboxyl groups has a molecular weight of about 40 to about 300 kDa, or from about 60 to about 160 kDa, or about 80 to about 120 kDa, or about 100 kDa.

8.-10. (canceled)

11. The pharmaceutical excipient composition of claim 1, wherein said functionalized anionic polysaccharide having carboxyl groups has a degree of substitution greater or equal to 0.15, or a degree of substitution of 0.7.

12. (canceled)

13. A monolithic solid dosage form for dual rate release of an active pharmaceutical ingredient, comprising the pharmaceutical excipient composition of claim 1 and said active pharmaceutical ingredient.

14. The monolithic solid dosage form of claim 13, further comprising a lubricating agent.

15. The monolithic solid dosage form of claim 14, wherein said lubricating agent is chosen from magnesium stearate, calcium stearate, sodium stearate, sodium lauryl sulfate, mineral oil, polyethylene glycol, glyceryl palmitostearate a wax, glyceryl behenate, liquid paraffin.

16. The monolithic solid dosage form of claim 13, further comprising a bulking agent.

17. The monolithic solid dosage form of claim 16, wherein said bulking agent is microcrystalline cellulose.

18. The monolithic solid dosage form of claim 13, further comprising a glidant.

19. The monolithic solid dosage form of claim 18, wherein said glidant is a talc, a silicone dioxide, or combinations thereof.

20. The monolithic solid dosage form of claim 13, further comprising a disintegrating agent.

21. The monolithic solid dosage form of claim 20, wherein said disintegrating agent is cross-linked povidone, cross-linked sodium carboxymethyl cellulose, sodium starch glycolate, or combinations thereof.

22. The monolithic solid dosage form of claim 13, further comprising a stabilizer.

23. The monolithic solid dosage form of claim 22, wherein said stabilizer is carboxymethyl starch, an amino acid, or combinations thereof.

24. The monolithic solid dosage form of claim 23, wherein said amino acid is arginine.

25. The monolithic solid dosage form of claim 13, wherein said active pharmaceutical ingredient is acetaminophen or ciprofloxacin.

26. (canceled)

27. A method of treatment of a bacterial infection comprising administering to a subject in need thereof a therapeutic amount of a monolithic dosage form according to claim 25, comprising ciprofloxacin.

28.-39. (canceled)
